(19) United States
(12) Patent Application Publication
(10) Pub. No.: US 2006/0024803 A1
(43) Pub. Date: Feb. 2, 2006

(54) Method and device for ultrasonic inoculation of biological cell material

(75) Inventors: Hans-Peter Berlien, Berlin (DE); Gerhard Muller, Berlin (DE); Carsten Philipp, Berlin (DE); Peter Urban, Berlin (DE)

Correspondence Address: KING & SPALDING LLP, 191 PEACHTREE STREET, N.E., 45TH FLOOR, ATLANTA, GA, 30303-1763, US

(73) Assignee: Dornier MedTech Systems GmbH, Wessling (DE)

(21) Appl. No.: 10/644,971

(22) Filed: Aug. 19, 2003

Related U.S. Application Data
(63) Continuation of application No. PCT/DE02/00581, filed on Feb. 18, 2002.

(30) Foreign Application Priority Data
Feb. 19, 2001 (DE) 101 08 799.3

(51) Int. Cl. C12N 13/00; C12M 1/00

(52) U.S. Cl. 435/173.5; 435/285.1; 366/127

(57) ABSTRACT

A method and device are provided for the ultrasonic inoculation of biological cell material. The method and device can be implemented by transmitting ultrasonic energy via a device, that includes an ultrasonic transducer and suitably formed glass fibres coupled thereto, into the immediate vicinity of the cells to be inoculated in a fluid containing the inoculation medium or a tissue aggregation. A simple method for low-cost inoculation of individual cells or cell aggregations with biological molecules and/or with pharmaceutical particles is thereby provided.

### CROSS-REFERENCE TO RELATED APPLICATION

This application is a continuation, under 35 U.S.C. § 365(c), of the co-pending PCT patent application having International Application No. PCT/DE02/00581, International Filing Date 18 Feb. 2002 (18.02.02), and Priority Date 19 Feb. 2001 (19.02.01), which claims priority to German Patent Application No. 101 08 799.3, filed on Feb. 19, 2001, and which is entirely incorporated herein by reference. Therefore, this application claims the benefit of the Feb. 19, 2001 filing date of German Patent Application No. 101 08 799.3, based on the foregoing chain of co-pendency.

### FIELD OF THE INVENTION

The invention generally relates to the application of ultrasonic energy to biological cell material, and more particularly, to a method and device for ultrasonic inoculation of biological cell material.

### BACKGROUND OF THE INVENTION

It is known to locally open individual cells by means of injection needles and/or laser beams via mechanically or optically controlled micromanipulators and to introduce through the channels created in this way biological or pharmaceutical material into the cells. In this respect, a large number of publications and patent applications exist, which are known to the person skilled in the art who takes an interest in such methods. However, all these methods have in common that the course of action which has to be taken for opening the cell necessitates the use of extremely complicated precision technology and that the costs for an inoculation of a single cell are therefore very high.

### SUMMARY OF THE INVENTION

Various embodiments of the present invention are directed to a method and a device for inoculating individual cells, cell ensembles, or even tissue aggregations, with the desired biological or pharmacological material as easily as possible and with high efficiency. According to various embodiments of the invention, it is possible to influence the cell membranes by locally induced ultrasonic oscillations in such a way that they become more permeable under the influence of this ultrasonic action and, especially, to open individual pores, existing in the cells by nature, to such an extent that biological or pharmacological material which is present in the surroundings of the respective cell can penetrate into the cell. This process can be particularly preferable in the presence of cavitation. Further, according to various embodiments of the invention, it is possible to transmit by means of suitably dimensioned, flexible glass fibres, and fibre bundles, respectively, ultrasonic oscillations in the frequency range of from above 20 kHz up to approximately 50 to 100 MHz. Moreover, depending on the viscosity of the surroundings of the fibre tip, cavities may be formed at ultrasonic frequencies between 20 and 100 Hz, wherein the resultant cavitation dynamics supports and/or provides a very useful condition for the process of introducing biological and pharmacological material into the cells that are present in the sound field.

In accordance with one aspect of the invention, one or a plurality of individual ultrasound-carrying glass fibres can be introduced into a suspension of cells and inoculation material in a solution and excited through electric or magnetostrictive ultrasonic generators, which are coupled thereto in a suitable manner. In this regard, an exemplary arrangement, which can be particularly suitable, is an array of a piezoelectric compound transducer defining together with the glass fibre or the fibre bundle an acoustic system that is excited in resonance. Sound can be advantageously coupled into the glass fibre or the fibre bundle by a mechanical connection, established, for example, by means of an adhesive or clamping at a point at which the amplitude of the mechanical stress is very small (i.e., a stress node). The length of the glass fibre or the fibre bundle in such case preferably corresponds to a multiple of half the wavelength. Since the process of a transmembrane inoculation of cells can be particularly effective in the threshold region of cavity formation, the acoustic system can comprise an ultrasound detector which can, via a feedback measurement of the developing ultrasonic standing wave field, detect the point where cavitation at the distal end begins and which controls, in accordance therewith, the amplitude and, where appropriate, the frequency of the oscillator in a feedback mode.

In accordance with another aspect of the invention, the method for inoculating individual cells or cell aggregations can also be used for medical applications in such a way that inoculation material can be introduced via a guide catheter into the target region of a biological tissue and that the ultrasound-carrying glass fibre can be introduced via the same catheter or a second access means, whereupon an ultrasonic field can build up in the area where cavitation begins in the target region to be treated, so as to promote in this way faster inoculation of the target tissue material on a cellular level with biological/genetic or pharmacological material.

### BRIEF DESCRIPTION OF THE DRAWINGS

The accompanying drawings are incorporated into and form a part of the specification for the purpose of explaining the principles of the invention. The drawings are not to be construed as limiting the invention to only the illustrated and described examples of how the invention can be made and used. Further features and advantages will become apparent from the following and more particular description of the invention which is illustrated in the accompanying drawings, wherein:

FIG. 1 is a schematic diagram illustrating a device according to various embodiments of the present invention.

FIG. 2 is a schematic diagram illustrating another device according to various embodiments of the present invention.

### DETAILED DESCRIPTION OF THE INVENTION

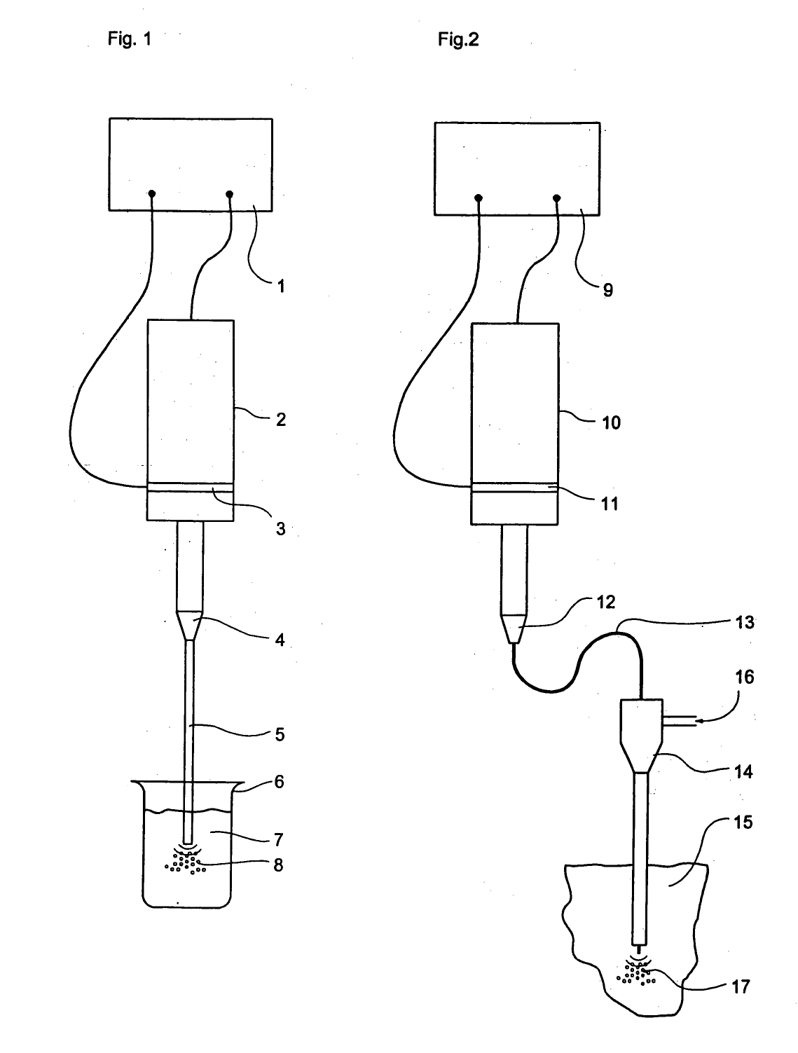

The illustrated embodiments of the present invention will be described with reference to the figure drawings wherein like elements and structures are indicated by like reference numbers. Referring now to FIGS. 1 and 2, the principle of the device according to exemplary embodiments of the present invention is explained in detail. FIG. 1 shows a device, according to various embodiments of the present invention, comprising an ultrasonic transducer 2 provided with a device 3 for measuring the amplitude, which may e.g. be defined by an additional, passive piezo disk, and the mechanical coupling means 4 for the glass fibre 5. The ultrasonic transducer 2 can be driven by the electric ultrasonic generator 1, which can simultaneously evaluate the signal of the measuring device 3 and control the frequency and the amplitude so as to obtain an optimum effect. The distal end of the fibre 5 can be immersed in the suspension 7 of the cells and the inoculation material in solution, which can be contained in the reaction vessel 6. The cavitation effects 8 that can be produced by the ultrasound in the suspension 7 can allow or support the introduction of the biological or pharmacological material into the cells.

FIG. 2 shows a device, according to various embodiments of the present invention, that can inoculate cells in a tissue aggregation. The ultrasonic transducer 10 provided with the amplitude measuring device 11 and the coupling means 12 can transmit an ultrasonic oscillation to the flexible glass fibre 13. The ultrasonic transducer 10 can be driven by the generator 1, which, with the aid of the signal of the measuring device 11, can simultaneously control the amplitude and the frequency to a value producing the optimum effect. The glass fibre 13 can extend through a guide catheter 14 into the tissue area 15 to be treated. The biological or pharmacological inoculation material in solution can be injected 16 through the guide catheter 14, wherein the ultrasonic effects 17 can allow this material to penetrate into the cells.

While the invention has been described with respect to the foregoing exemplary embodiments, it will be apparent to those skilled in the art that various modifications, variations and improvements of the invention can be made in light of the above teachings and within the purview of the appended claims without departing from the spirit and intended scope of the invention. In regard to the foregoing description of the exemplary embodiments of the invention, areas which are known to those of ordinary skill in the art have not been described in detail in order to facilitate a clear and concise description of the invention. Accordingly, it should be understood that the invention is not to be limited by the specific exemplary embodiments, but only by the scope of the appended claims.

What is claimed is:

1. A device for effecting ultrasound-assisted inoculation of cells, comprising:
an ultrasonic transducer; and
a glass fibre coupled to the ultrasonic transducer, wherein the glass fibre is configured to transmit ultrasonic energy into a vicinity of cells to be inoculated in a fluid containing an inoculation medium.

2. The device of claim 1, further comprising a measuring device coupled to the glass fibre and configured to detect by ultrasonic impedance measurement the beginning of cavitation by the glass fibre.

3. A device for effecting ultrasound-assisted inoculation of cells in a tissue aggregation, comprising:
an ultrasonic transducer;
a flexible glass fibre coupled to the ultrasonic transducer; and
a catheter through which the flexible glass fibre extends, wherein the flexible glass fibre is configured to transmit ultrasonic energy into a vicinity of cells to be inoculated in a tissue aggregation.

4. The device of claim 3, further comprising a measuring device coupled to the flexible glass fibre and configured to detect by ultrasonic impedance measurement the beginning of cavitation by the flexible glass fibre.

5. A method for effecting ultrasound-assisted inoculation of cells, comprising:
providing a device comprising an ultrasonic transducer and a glass fibre coupled to the ultrasonic transducer; and
transmitting ultrasonic energy via the glass fibre into a vicinity of cells to be inoculated in a fluid containing an inoculation medium.

6. The method of claim 5, wherein providing a device further comprises providing a measuring device coupled to the glass fibre and configured to detect by ultrasonic impedance measurement the beginning of cavitation by the glass fibre.

7. A method for effecting ultrasound-assisted inoculation of cells, comprising:
providing a device comprising an ultrasonic transducer, a flexible glass fibre coupled to the ultrasonic transducer, and a catheter through which the flexible glass fibre extends; and
transmitting ultrasonic energy via the flexible glass fibre into a vicinity of cells to be inoculated in a tissue aggregation.

8. The method of claim 7, wherein providing a device further comprises providing a measuring device coupled to the flexible glass fibre and configured to detect by ultrasonic impedance measurement the beginning of cavitation by the glass fibre.
